(19) United States
(12) Patent Application Publication
(10) Pub. No.: US 2008/0241860 A1
(43) Pub. Date: Oct. 2, 2008

(54) METHODS AND APPARATUS TO RECOGNIZE NERVE INJURIES

(76) Inventor: Klaus J. Korak, Holn (DE)

Correspondence Address: HANLEY, FLIGHT & ZIMMERMAN, LLC, 150 S. WACKER DRIVE, SUITE 2100, CHICAGO, IL, 60606, US

(21) Appl. No.: 12/101,662

(22) Filed: Apr. 11, 2008

Related U.S. Application Data
(63) Continuation of application No. PCT/IB2006/003854, filed on Oct. 11, 2006.
(60) Provisional application No. 60/725,476, filed on Oct. 11, 2005.

(51) Int. Cl. G01N 33/566

(52) U.S. Cl. 435/7.2

(57) ABSTRACT

Methods and apparatus are described to recognize nerve injuries, specifically brachial plexus injuries, and their severity, preferably of newborn children. To achieve an inexpensive and quick test, which can be executed without harming the patient, it is proposed to measure the motor neuron loss by measuring a biochemical marker, i.e. NSE or protein S100 in a body fluid.

### RELATED APPLICATIONS

This patent is the National Stage of International Patent Application Serial No. PCT/IB2006/003854, filed October 11, 2006, which claims priority to U.S. Provisional Patent Application Ser. No. 60/725,476, filed on Oct. 11, 2005, both of which are hereby incorporated herein by reference in their entireties.

### FIELD OF THE DISCLOSURE

This disclosure relates generally to medical devices, and, more particularly, to methods and apparatus to recognize nerve injuries, specifically brachial plexus injuries and their severity.

### BACKGROUND

Anatomy and Mechanism of Injury

The brachial plexus (as used herein, the term “brachial plexus” refers to a large network of nerves that originates at the spinal cord and distributes the motor and sensory innervation to the upper extremity) is a fragile group of nerves composed of the four cervical spinal roots C5, C6, C7, and C8, and the first thoracic root T1 (FIG. 1). The nerves that form the brachial plexus originate from the ventral and dorsal horn of the spinal cord and exit through the vertebral foramen. The large network of nerves—the brachial plexus—extends from the spinal cord to the axilla. In the axilla the brachial plexus divides to form the peripheral nerves that provide the motor and sensory innervation of the upper extremities.

During passage through the birth canal, excessive traction can be exerted on the neck, widely separating head and shoulder and therefore putting excessive stress on the brachial plexus. As a result the nerves of the brachial plexus may be injured (FIG. 2).

Incidence and Risk Factors

Brachial plexus injuries are the second most frequent form of birth trauma, surpassed in number only by fractures of the clavicle. The reported incidence of obstetric brachial plexus palsy (as used herein, the term “obstetric brachial plexus palsy” or “obstetric plexopathy” refers to the full clinical picture that may develop after brachial plexus birth injuries including, but not limited to, functional disabilities, a shortened upper extremity on the affected side, bone deformities and joint contractures, hereinafter referred to as OBPP) in the United States ranges from 0.38 to 2.6 per 1000 live births. This rate is most likely lower than the actual number, because in some children symptoms resolve spontaneously. These children are typically never presented to a specialist for consultation.

Predisposing factors include large birth-weight babies, maternal multiparity, vertex presentation, delivery assisted by instruments or vacuum. The common denominator of all these risk factors is the increased possibility of a difficult passage through the birth canal, thus predisposing the brachial plexus to traction forces. In addition to very large babies, very small babies are at risk. In these small, mostly pre-term babies, the brachial plexus is not as well protected from excessive forces and maneuvers. Another interesting finding is that the increase of Caesarean sections has not reduced the overall incidence of brachial plexus injuries.

Initial Diagnosis

Although some infants with brachial plexus injuries recover spontaneously or have only minor residual functional deficits, about a quarter of affected children have persistent functional limitation. In the most severe cases, shoulder, arm and hand function are permanently impaired. Motor and sensory deficits, the resulting muscle imbalance and abnormal stresses on joints and bones lead to severe forms of progressive bone deformities and joint contractures.

Defining the Problem

A child with a suspected brachial plexus lesion should be examined as early as possible to reach a definite diagnosis, to begin the recording of the natural progression of recovery, and to initiate education and support of tie family. The diagnosis may be evident at birth. After difficult delivery of a very large or very small baby, the upper extremity may be flailing and dangling. After 48 hours, muscle testing allows the determination of two basic clinical types. An injury to the upper roots of the brachial plexus (C5/C6 or extended as C5/C6 and C7) results in a lesion pattern characterized by an arm that is held in internal rotation and pronation. Elevation of the arm in the shoulder joint is impossible, while hand function is normal (FIG. 3). The picture of complete paralysis is different. The entire arm is flailed and the hand clutched, without any tonus. Infants have a vasomotor impairment and some show a positive Horner sign. During the first weeks, infants are closely monitored, and progress is recorded (to the extent progress exists). Clinical intervention in these first weeks is limited to mild conservative therapy (i.e. passive movement of the extremity).

Defining the Problem

The evaluation of infants includes the investigation of shoulder (abduction/external rotation), elbow (flexion/extension), wrist and hand (flexion/extension/thumb adduction/abduction), in addition to a comprehensive neurological and clinical examination. While the initial diagnosis is straight forward—as it reflects the clinical status of the extremity—consensus on further clinical management of the infant is lacking.

According to Birch 2002 (Berger R P et al., “Serum Neuron-Specific Enolase, S100B, and Myelin Basic Protein Concentrations After Inflicted and Noninflicted Traumatic Brain Injury in Children,” J. Neurosurg: Pediatrics,vol. 103, July 2005) and Birch R. (“Invited editorial: Obstetric Brachial Plexus Palsy,” J. Hand Surg., 2002, 27B: 3-8), the ambiguity does not include the extreme forms of brachial plexus injury, i.e., a very mild and a very severe form of injury. In these cases, prognosis and treatment options are relatively clear: mild forms of brachial plexus injury show signs of functional recovery within days and therefore require no, or only mild conservative treatment, whereas the severe forms require surgical exploration and reconstruction of the brachial plexus. The optimal time-point for surgery—though still under debate—is thought to be between three and six months of age.

In the greater part of the affected infants, however, clinical evaluation does not allow a precise assessment of the injury. An issue is the absence of objective parameters that allow an initial, immediate, and objective assessment of the injury's extent. The objective assessment of injury serves as the necessary basis for therapeutic planning, precise prognosis and reliable prediction of the functional end stage. Today, a frequently encountered problem is that children who initially present identical clinical symptoms develop a completely different outcome. Here, reliable indicators for prognosis are missing.

According to van Dijk 2001 (Van Dijk J G et al., “Obstetric Lesions of Brachial Plexus,” Muscle Nerve 2001, 24: 1451-1461), treatment in these infants cannot be based on objective and accepted methods (i.e., “state of the art”), but depends on the experience and subjective opinion of the individual physician. In this situation, some physicians agree that attempts to avoid the sequelae of late plexus palsy necessitate surgical intervention early in the first year, while others refrain from surgery completely. Still others advocate a wait-and-see approach, reserving surgery for infants in whom spontaneous recovery is deemed insufficient after a certain period, the length of which is itself a matter of debate. The lack of consensus among the medical community is due to the fact that contemporary techniques beyond clinical investigation are still vague, ambiguous, and imprecise. The diagnostic armament—beyond clinical investigation—is limited to imaging techniques and electromyography.

Clinical Investigation

Evaluation of the child is difficult and requires an experienced examiner. But even for the experienced, examinations are difficult to accomplish, as functional evaluation in newborns has major drawbacks. First, voluntary movement needs to be possible upon command. This, however, is first possible at three to four years of age. At the same time, the degree of functional development at a specific time point varies among newborns. In addition to the lack of cooperation, motor control in newborns is immature. Less severe lesions may only become apparent when motor control becomes sufficiently subtle to require the full range of movement. When the function under observation is not, or is only partially developed, clinical assessment is severely limited. As a result uniform testing paradigms for children are difficult to establish. At the same time, the different significance given by each author to the appraisal of clinical results adds to the confusion. Matters aggravated by the fact that in obstetric plexopathy the affected arm may grow less than the normal one. This contributes to the functional handicap that cannot be assessed at an early age. Furthermore, persistent palsy is also associated with psychosocial problems not yet present in infants. A functional end stage can only be assessed reliably in children of about four years or older.

Imaging Studies

Until the advent of MRI, CT myelography was the standard method for evaluating the integrity of the brachial plexus, and it remains arguably the most sensitive radiographic study to detect nerve root injuries. A water-soluble dye is injected intrathecally, and CT images of the area in question are obtained. The main drawbacks of the procedure are the radiation exposure, the need for sedation, significant false-positive rate, and the lack of information on the distal brachial plexus. Some have abandoned the use of CT myelography because direct observation during surgical exploration did not always correlate with CT myelographic findings. Today, high-resolution MRI is the best imaging study available for evaluating neonatal brachial plexus palsy (hereinafter referred to as BPP). MRI requires no radiation exposure, is noninvasive, and provides more detail than CT myelography. According to Birch 2002, this test is most useful preoperatively and is most often used in the most severe cases to verify a suspected root avulsion. According to Van Dijk 2001, in the large number of moderate cases with similar clinical symptoms, MR imaging does not provide the additional information, necessary to further differentiate among the individual children.

Electromyography

According to Van Dijk 2001, electromyography (EMG) is a neurophysiologic test that examines the electrical activity in muscle units and looks for evidence of denervation or reinnervation. The initial study usually is performed two to tree weeks after injury, when signs of denervation are seen in children with moderate or severe injuries. Electro diagnostic studies are used as an extension of the physical examination and can provide data on the severity of the injury. Tie usefulness of EMG and other electro diagnostic procedures has been recently questioned by numerous authors, as it is difficult to employ EMG in infants with sufficient reliability. Some authors feel that EMG provides useful information to track the reinnervation process and guide in surgical decision-making. Others believe that the EMG does not provide prognostic information and consider electromyography unreliable because EMG predictions frequently have no clinical correlation. Therefore, the place for neurophysiologic examinations remains controversial.

Conclusion

In conclusion, controversies arising from obstetric brachial plexus birth injuries are numerous. They include:
(1) the question whether conservative or surgical treatment will yield the best result;
(2) the prediction of the best time point for surgical intervention;
(3) the difficulty in precise clinical evaluation;
(4) difficulty in precise electro diagnosis and magnetic resonance image reading;
(5) the lack of an uniform evaluation system;
(6) lack of consensus what constitutes a good or poor result;
(7) lack of reliable data on the initial clinical picture; and
(8) the pattern of recovery varies in individual infants.
   In this context, the clinician cannot arrive at a precise diagnosis and an unambiguous prognosis when faced with a baby with injury to the brachial plexus.

### BRIEF DESCRIPTION OF THE DRAWINGS

FIG. 1 is an illustration showing the brachial plexus.

FIG. 2 is an illustration of a brachial plexus of a baby being injured during birth.

FIG. 3 is an illustration of a child with a late obstetric brachial plexus palsy.

FIG. 4 shows a target-dependency of immature motor neurons.

FIG. 5a is horizontal cross-sectional view through a lateral motor column of a sixth cervical segment of a specimen (a five day old Sprague-Dawlyte rat) showing an injury-induced motor neuron loss after injury to the brachial plexus at birth on a lesioned side.

FIG. 5b is a horizontal cross-sectional view through a lateral motor column of a sixth cervical segment of the specimen of FIG. 5a on an intact side and showing the deleterious effects of nerve injury at birth on motor neurons of the lateral motor column when compared to FIG. 5a. For FIGS. 5a and 5b: H&E, ×100; Scale bar in FIG. 5b=100 μm.

FIG. 5c is an image of motor neurons with clearly visible nucleoli on the healthy side

FIG. 5d is an image of psychotic nuclei of dying motor neurons after crush injury. For FIGS. 5c and 5d: H&E, ×400; Scale bar in FIG. 5d=20 μm. Permanent motor neuron loss limits axonal regeneration as the basis for functional recovery.

FIG. 6 is a chart showing a time dependency of motor neuron loss.

FIG. 7a is a schematic diagram showing an intact motor neuron-muscle interaction.

FIG. 7b is a schematic diagram showing an injury that disrupts motor neuron-muscle interaction.

FIG. 7c is a schematic diagram showing shows a degenerating motor neurons release a diverse number of substances into the surrounding environment.

FIG. 8a is a chart showing an increase in marker quantity in newborn (BLACK) as compared to the normal population (WHITE) as an indicator for motor neuron degeneration in the spinal cord.

FIG. 8b is a chart showing a decrease in marker quantity in newborn (BLACK) as compared to the normal population (WHITE) as an indicator for motor neuron degeneration in the spinal cord;

FIG. 8c is a chart showing the presence of a marker in newborn (BLACK) that is absent in the normal population (WHITE) as an indicator for motor neuron degeneration in the spinal cord.

FIG. 9 is a chart showing a marker quantity in newborn (BLACK) reveals a more severe injury to the spinal motor neuron pool as compared to newborn (WHITE) and the normal population.

FIG. 10 shows a schematic view on a preferred example OBPP Triple Source Test Kit.

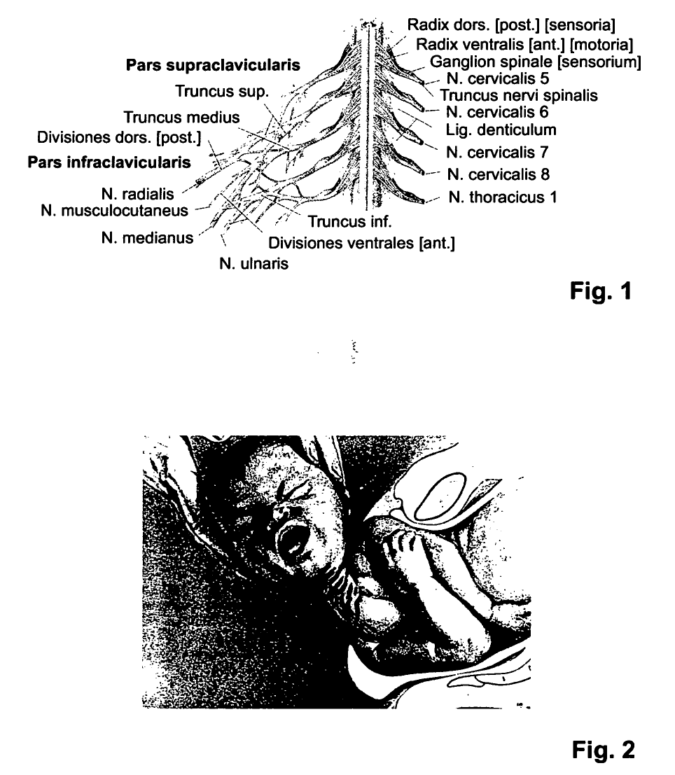

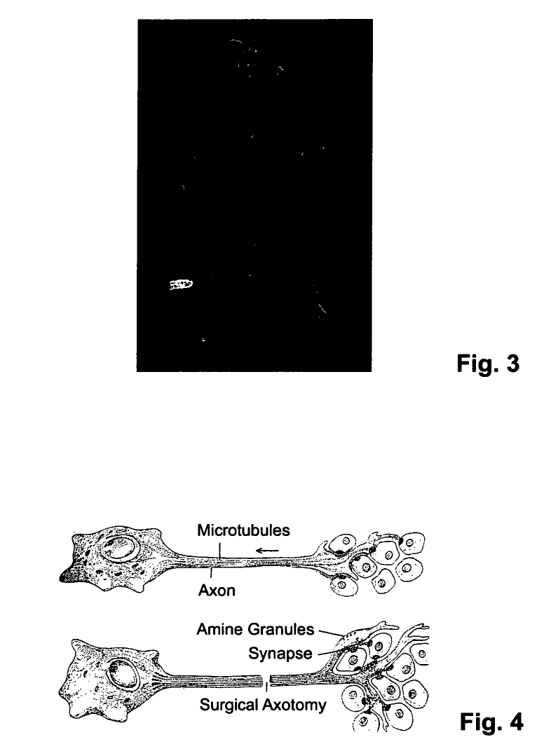

### DETAILED DESCRIPTION

The example methods and devices described herein recognize nerve injuries, especially brachial plexus injuries at an early stage of life of a child, avoiding the disadvantages of MM, CT or EMG. The example methods and devices described herein may also be used to realize the severity of nerve injuries, especially brachial plexus injuries at an early stage of life of a child again, avoiding the disadvantages of MRI, CT or EMG.

The example method described herein recognizes nerve injuries, especially brachial plexus injury, by measuring the motor neuron loss. Furthermore, the example methods may be used to determine the severity of nerve injuries, especially brachial plexus injury, by measuring the motor neuron loss and by comparing the result with a reference value.

Also, as described herein, the presence of a biochemical marker, especially NSE or protein S-100, is measured in a body fluid, for example, serum, blood, urine, plasma or cerebrospinal fluid, taken from the patient, which allows fur a quick and inexpensive determination of a brachial plexus injury. Furthermore, the example methods and devices described herein may be performed/used anywhere because the example device is very small and light and a laboratory is no longer necessary. In addition, testing for an injury through the example methods is very harmless for the patient because he only has to give a little sample of body fluid.

Spinal Cord Injury

Since Smellie's first clinical description of OBPP in 1786, concepts of pathology and treatment strategies have focused on the injury of the peripheral nerve. Investigations of central nervous events in injury and recovery were not pursued, even though Boyer had already described the dramatic central nervous effects of brachial plexus birth traumas in humans as early as 1911.

It is of significance that injury strikes an immature nervous system. During the early postnatal period interaction between maturing motor neurons and their target structures is vital for motor neuron survival (FIG. 4). FIG. 4 shows a target-dependency of immature motor neurons.

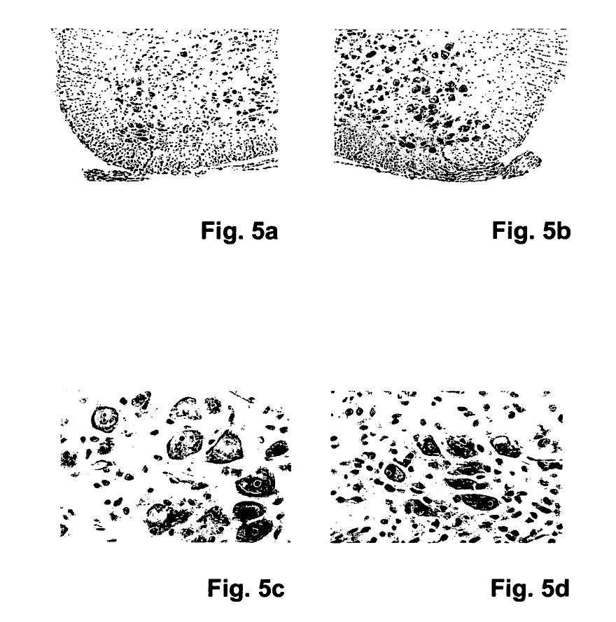

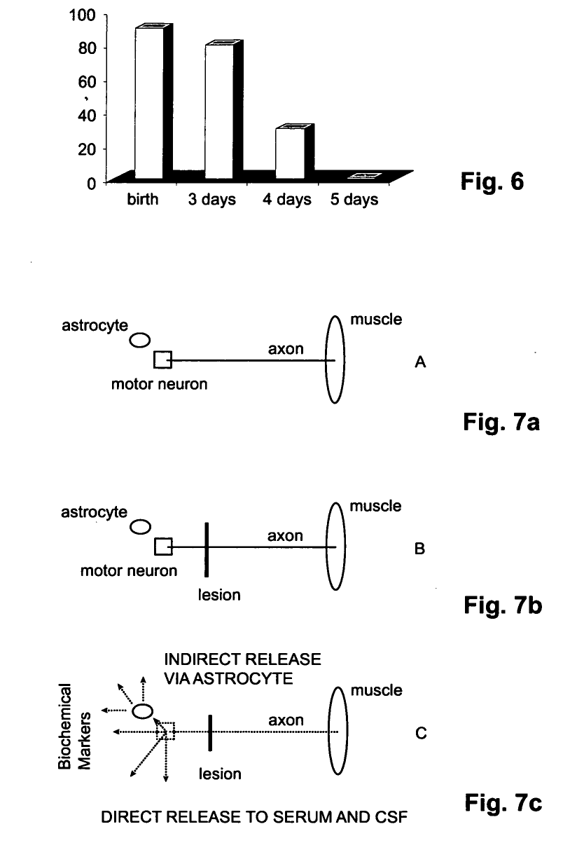

Axon injury during this critical period initiates events in corresponding motor neurons, ultimately leading to their death. This motor neuron loss is an essential feature of any nerve injury in the early postnatal period, because feedback between motor neurons and their target muscle is critical for motor neuron survival during this vulnerable period. As motor neurons mature they gradually lose their dependency of muscle-derived neurotrophic factors. Therefore, motor neuron loss is reduced to 50% when nerve transactions are performed one week after birth, and becomes nearly undetectable when axotomies are performed in one month-old animals. Time dependency of motor neuron loss is well characterized in numerous experimental models (FIG. 5).

As detailed above, FIG. 5a is horizontal cross-sectional view through a lateral motor column of a sixth cervical segment of a specimen (a five day old Sprague-Dawlyte rat) showing an injury-induced motor neuron loss after injury to the brachial plexus at birth on a lesioned side. Similarly, FIG. 5b is a horizontal cross-sectional view through a lateral motor column of a sixth cervical segment of the specimen of FIG. 5a on an intact side and showing the deleterious effects of nerve injury at birth on motor neurons of the lateral motor column when compared to FIG. 5a. In addition, FIG. 5c is an image of motor neurons with clearly visible nucleoli on the healthy side, and FIG. 5d is an image of psychotic nuclei of dying motor neurons after crush injury. Permanent motor neuron loss limits axonal regeneration as the basis for functional recovery.

Whereas every axonal injury in adults leads to characteristic neural and non-neural reactions in the spinal cord, the fundamental feature of a proximal nerve injury in newborns—the dramatic motor neuron loss—is absent. Based on clinical and experimental evidence, motor neuron loss in the spinal cord is a distinctive feature of any proximal nerve injury in the immature newborn. Because recovery of motor and sensory function is attributed to axonal regeneration followed by original target reinnervation, it becomes apparent that the extent of motor neuron loss is a limiting factor for recovery of function. Loss of motor neurons eliminates all chances of recovery based on axonal outgrowth and reinnervation.

Direct trauma to the nerves of the brachial plexus at birth results in an injury-induced motor neuron loss in corresponding motor neuron pools in the spinal cord. The motor neuron loss limits recovery and therefore becomes a fundamental component of OBPP pathology.

Biochemical Indicators

Motor neurons are structural entities (FIG. 7a). Independent of the mechanism, motor neuron degeneration compromises their structural integrity (FIG. 7b). A break-down of the cell membrane is followed by a release of numerous substances into the surrounding tissue. Motor neuron components, set free after cell disintegration, induce cells in the surrounding environment, i.e., the astrocyte, to release further substances into the environment (FIG. 7c). The qualitative and quantitative pattern of these substances in, for example, serum, cerebrospinal fluid, or urine can be detected early after a brachial plexus birth injury. Qualitative and quantitative analysis of serum and CSF composition in the early postnatal period can be used as an objective diagnostic and prognostic tool in children with obstetric brachial plexus injuries.

Biochemical Markers in Clinical Practice

Biochemical markers may be integrated into a clinical routine, for example, as follows:
The infant presents clinical symptoms of brachial plexus injury at birth.
Blood, cerebrospinal fluid, and/or urine samples are collected, for example, at 2 h, 6 h, 12 h, and 24 h after birth.
The OBPP TRIPLE SOURCE test kit is employed.
A positive OBPP TRIPLE SOURCE Lest is confirmed with laboratory techniques.
An increase, decrease, and qualitative changes are established for the various time points.
Positive predictive value, negative predictive value, sensitivity, and specificity for development of mild moderate and severe forms of OBPP are obtained using optimal cut-off levels and are calculated.
Standard neurological examinations are performed at, for example, the equal time points.

Possible motor neuron death—marker relationships may include:
Motor neuron death correlates directly to marker concentration. An increase in motor neuron death is mirrored in an increase of marker release.
Motor neuron death correlates inversely to marker concentration. An increase of motor neuron death is mirrored in a decrease of marker concentration.
Motor neuron death alters the qualitative composition of serum and cerebrospinal fluid. Motor neuron death results in the presence of markers normally absent in serum and cerebrospinal fluid.
Motor neuron death alters tie qualitative composition of serum and cerebrospinal fluid. Motor neuron death results in the absence of markers normally present in serum and cerebrospinal fluid.

Integration of Motor Neuron Death, Biochemical Markers and Clinical Assessment

Case Study A:

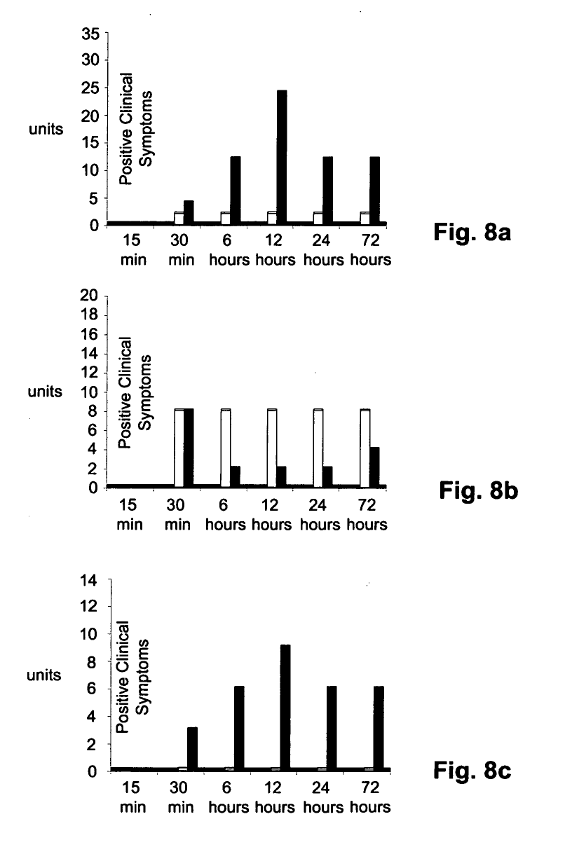

Turning to FIGS. 8a-c, a newborn (BLACK) shows clinical symptoms of a brachial plexus injury at birth. Quantification of biochemical markers (FIGS. 8a/8b/8c) reveals a distinct marker profile at defined postnatal time-points as compared to the normal population (WHITE). The biochemical markers collected from serum, urine and/or cerebrospinal fluid may include, but are not limited to, neuron-specific enolase (NSE), protein S100B, cytokines (e.g., interleukins IL-6, IL-8, IL-10) neurotrophins (e.g., NOF, NT-3, NT-4, NT-5), glial fibrillary acidic protein, excitatory amino acids (e.g., glutamate, quinolinic acid), and glial-cell-derived neurotrophic factor GDNF. Each biochemical marker has its own distinct profile in normal children and children with obstetric brachial plexus lesions. Possible profile pattern include an increase (FIG. 8a), decrease (FIG. 8b), and/or the presence of normally absent biochemical markers (FIG. 8c) as indicators of motor neuron degeneration in spinal cord.

Case Study B:

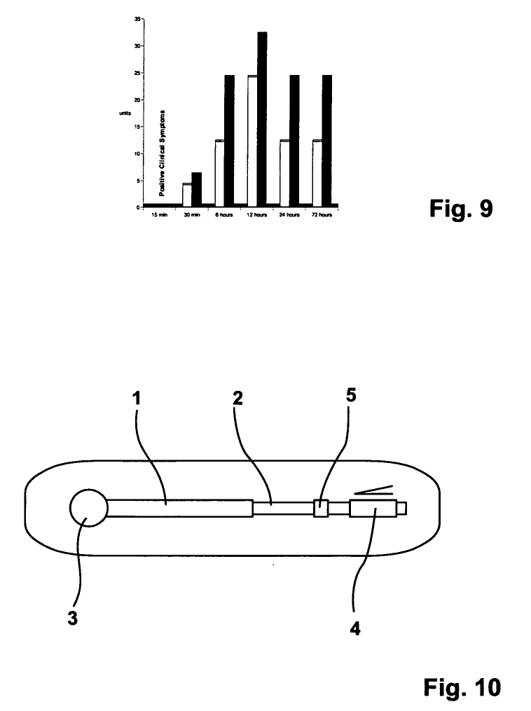

Turning to FIG. 9, two newborns (WHITE and BLACK) show identical symptoms of a brachial plexus injury at birth. Analysis of biochemical markers (vide supra) (FIG. 9) uncovers a significantly greater increase in marker quantity at distinct postnatal time points in newborn (BLACK) than in newborn (WHITE) and as in the normal population (for normal population see FIG. 8a). Analysis and comparison of marker quantity reveals a more severe trauma to the spinal motor pool in newborn (BLACK) than in newborn (WHITE) with the respective prognostic and therapeutic consequences. Biochemical markers are the first objective, observer-independent parameter in the diagnosis of brachial plexus birth injury. Because they correlate with the degree and extent of nerve injury, they allow a precise assessment of lesion extent. Because early indicators of OBPP are urgently needed to initiate optimal therapeutic intervention, serological detection of biochemical markers such as neuron-specific-enolase (NSE), protein S-100B and numerous secondary mediators of cell damage, i.e. glutamate, may provide a valuable diagnostic and prognostic tool in clinical management of early stages of OBPP.

Biochemical Markers as Indicators of Brain Damage

Biochemical markers as diagnostic and prognostic indicators are used in a variety of clinical situations (Berger 2002). Two proteins that have been frequently employed as primary biochemical indicators of neuron damage are NSE and protein S100. In addition to these primary indicators, mediators of secondary damage such as excitatory amino acids (glutamate, quinolinic acid), cytokines (interleukin 6, 8, 10), and markers of delayed neuronal death (nucleosomes, cytochrome-C) were investigated in a number of experimental and clinical settings (Berger 2002). In a study it was reported that CSF levels of glutamate were massively increased for a prolonged period of time in newborns and children with traumatic brain injury. Others recently observed that increases in the apoptosis trigger cytochrome-C were associated with traumatic brain injury and mortality. Neuron-specific enolase (NSE), the neuronal form of the intracytoplasmatic glycolytic enolase, is an enzyme primarily localized in the neuronal cytoplasm. It is found in neuronal cell bodies, axons and neuroendocrine cells and in neuroendocrine tumors. Previous immunohistochemical studies in animals and humans revealed an early release of NSE by damaged neurons, thereby indicating functional disturbances or structural defects of are plasma membrane. Review of the literature shows several experimental studies on variations in NSE levels in the CSF after global ischemia, stroke or trauma. Moreover, cerebrospinal fluid NSE was assayed in humans suffering from stroke, transient ischemic attacks, intracerebral hematoma, subarachnoid hemorrhage, head injuries, cardiac arrest, pediatric encephalitis, acute encephalopathy, and Reye syndrome. The essential findings of both experimental and clinical studies demonstrated that cerebrospinal fluid NSE is a sensitive and reliable indicator of even minor brain injuries and shows good correlation with the severity of the resulting lesion. Protein S-100 is a dimeric acidic calcium binding protein, expressed primarily in astroglial cells. Protein S-100B (beta-beta-subunits) and S-100A1 (alpha-beta subunits) are predominantly present in astrocytes and Schwann cells. It physiologic function is not completely understood, but its levels are increased in the presence of central nervous system lesions. There is a significant correlation between early S-100B values and the volume of cerebral contusion in patients with TBI.

Neurotrophins are a family of structurally related molecules that play important roles in survival and differentiation of specific neuronal populations. Nerve-growth factor (NGF), brain-derived neurotrophic factor (BDNF), neurotrophin-3 and NT-4/NT-5 belong to this gene family. Studies have shown that BDNF levels in cerebrospinal fluid (CSF) from asphyxiated human neonates were significantly higher than those from controls, while NGO was significantly lower. These studies demonstrate the possibility to use neurotrophin levels in CSF as a diagnostic tool in clinical management.

In the context of these studies, the use of biochemical markers as an objective tool for diagnosis and prognosis in cases of brachial plexus birth injuries is well justified.

OBPP Triple Source Test Kit

Principle and Design

The schematic view of a preferred example device, i.e., the OBPP TRIPLE SOURCE Test Kit, shown in FIG. 10 shows the principal components and a generic design of the example device. In case motor neuron damage or death occurs, biomarkers of neuronal injury (e.g., NSE, Protein S-100, etc.) in, for example, blood, cerebrospinal fluid and/or urine increase. The OBPP TRIPLE SOURCE Test Kit serves a rapid one-step assay that employs qualitative and quantitative changes of NSE and Protein S-100 indicators of neuronal damage or death.

The OBPP TRIPLE SOURCE Test Kit is a rapid one-step chromatographic immunoassay (CIA). In the case of the OBPP TRIPLE SOURCE Test Kit, an absorbent (1) overlaps with a membrane (2) that is impregnated with lyophilized colloidal conjugate of gold particles and monoclonal solid phase antibodies against biomarkers of neuronal injury (e.g., NSE, Protein S-100, etc.).

Samples are collected, for example, at 0.5, 6, 12 and 24 hours after birth. Then the sample of, for example, serum, cerebrospinal fluid, or urine is introduced onto the collector (3) of the OBPP TRIPLE SOURCE Test Kit. Tie sample proceeds through the absorbent (1) and then laterally onto the chromatographic membrane (2). As it contacts the membrane (2), the sample dissolves lie lyophilized conjugate. A positive reaction is based on the presence of NSE and/or Protein S-100 or their metabolites in serum, cerebrospinal fluid or urine of the patient. In a reactive sample (test “positive”), antigens will attach to antibodies in the colloidal solution. As the conjugate moves forward on the membrane (2), monoclonal antibodies affixed on the graded test zone will bind the NSF-gold conjugate complex, forming a colored line (4) (“Neuron Loss”). All samples will cause a colored line to appear in the control zone (5). This line is formed by the binding of polyclonal antibodies (anti-mouse IgG) affixed on the control zone (5) to the sample-colloidal gold conjugate. Presence of this line indicates that the test has been carried out correctly. The specificity of the immune reaction avoids cross-reactions with structurally related antibodies.

The presence of increased levels of NSE and protein-B100 in the tested sample reveal that injury to neurons of the central nervous system has occurred. The therapeutic response must be adapted accordingly.

Although certain example methods, apparatus and articles of manufacture have been described herein, the scope of coverage of this patent is not limited hereto. On the contrary, this patent covers all methods, apparatus and articles of manufacture fairly falling within the scope of the appended claims either literally or under the doctrine of equivalents.

What is claimed is:

1. A method to recognize a nerve injury comprising measuring a motor neuron loss.

2. A method as defined in claim 1, wherein the nerve injury is a brachial plexus injury.

3. A method as defined in claim 1, further comprising comparing the measured motor neuron loss with a reference value to provide a relationship of the measured motor neuron loss to the reference value to determine a severity of the nerve injury.

4. A method as defined in claim 1, wherein the motor neuron loss is measured by measuring a biochemical marker in a body fluid.

5. A method as defined in claim 4, wherein the biochemical marker is one or more of a neuron-specific enolase NSE) or Protein S-100.

6. A method as defined in claim 4, wherein the body fluid is one or more of serum, blood, plasma, cerebrospinal fluid, or urine.

7. A method as defined in claim 4, wherein a sample of the body fluid is taken from the patient and the sample is examined by using a mobile chromatographic immunoassay (CIA) or in a laboratory.

8. A method as defined in claim 7, whereas the sample is collected 0.5, 6, 12 and 24 hours after birth.

9. A method of diagnosing a brachial plexus injury comprising detecting a change in the composition of a biochemical marker in a body fluid.

10. A method as defined in claim 9, wherein the body fluid is one or more of serum, blood, plasma, cerebrospinal fluid, or urine.

11. A method as defined in claim 9, wherein the biochemical marker is at least one of neuron- specific enolase (NSE) or protein S-100.

12. A method as defined in claim 9, further comprising examining the brachial plexus nerves of a newborn.

13. A device to recognize a nerve injury comprising a chromatographic immunoassay (CIA).

14. A device as defined in claim 13, wherein the nerve injury is a brachial plexus injury.

15. A device as defined in claim 13 further comprising:
a membrane; and
an absorbent to overlap with the membrane, wherein a body fluid sample proceeds through the absorbent.

16. A device as defined in claim 15, wherein the membrane is impregnated with lyophilized colloidal conjugate of gold particles.

17. A device as defined in claim 16, wherein the body fluid sample dissolves the conjugate to produce a reaction.

18. A device as defined in claim 13, wherein the membrane is impregnated with a monoclonal solid phase antibody.

19. A device as defined in claim 18, wherein the monoclonal solid phase antibody is against one or more of NSE and/or protein S-100.

20. A device as defined in claim 19, wherein the body fluid sample reactions with the antibody and the reaction is indicative of the nerve injury.
